(19) United States
(12) Patent Application Publication
(10) Pub. No.: US 2007/0281873 A1
(43) Pub. Date: Dec. 6, 2007

(54) Lubricant Composition for Fluid Dynamic Bearing

(75) Inventor: Tahei Okada, Chiba (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Idemitsu Kosan Co., Ltd, Tokyo (JP)

(21) Appl. No.: 11/574,321

(22) PCT Filed: Aug. 25, 2005
(86) PCT No.: PCT/JP05/15425

§ 371 Date: Feb. 27, 2007

(30) Foreign Application Priority Data
Aug. 30, 2004 (JP) 2004-249705

(51) Int. Cl. C10M 107/20; C07C 41/00

(52) U.S. Cl. 508/579; 568/579

(57) ABSTRACT

A lubricating oil composition for fluid dynamic bearings which comprises 50 to 100% by mass of an ether compound comprising at least one ether bond and having 11 to 34 carbon atoms as the base oil and has a kinematic viscosity of at least 2.2 mm²/s at 100° C. is provided. The composition has excellent viscosity characteristics such as excellent fluidity at low temperatures and a small decrease in the viscosity in a high temperature range, exhibits lower evaporability, excellent energy saving property and excellent heat stability and is advantageously used for compact fluid dynamic bearings used under rotation at a high speed.

### TECHNICAL FIELD

The present invention relates to a lubricating oil composition for fluid dynamic bearings and more particularly, to a lubricating oil composition for fluid dynamic bearings which has a small viscosity, a great viscosity index, excellent fluidity at low temperatures, lower evaporability and an excellent energy saving property and is advantageously used for compact fluid dynamic bearings used under rotation at a high speed.

### BACKGROUND ART

Heretofore, ball bearings and roller bearings have been used as the bearing in motors mounted in hard disk drives and the like. However, since decreases in the size, vibration and noise are required for the motors, fluid dynamic bearings are recently developed and used in practical applications.

The progress in the decreases in the size and the weight, the increase in the capacity and the increase in the speed of information processing in image and audio instruments and personal computers is really amazing

In these electronic instruments, various rotating devices, for example, for driving magnetic disks and optical disks such as FD, MO, minidisks, compact disks, DVD and hard disks, are used, and improvement in the bearing indispensable for the rotating devices contributes to the decreases in the size and the weight, the increases in the capacity and the speed described above to a great degree.

Since a fluid dynamic bearing comprising a sleeve and a rotating shaft faced to the sleeve via a lubricating oil does not have ball elements, the fluid dynamic bearing is suitable for decreasing the size and the weight, suppresses generated noise and is excellent in economy. The demand for the fluid dynamic bearing is increasing in the field of personal computers, audio instruments, visual instruments, mobile communication instruments and automobile navigation instruments.

For the lubricating oil used in the fluid dynamic bearing, excellent viscosity characteristics such as a small viscosity in a low temperature range, an excellent fluidity at low temperatures and a small decrease in the viscosity in a high temperature range and lower evaporability are required.

It is considered that a further increase in the speed of processing information in an increasing amount and further decreases in the size and the weight of the instruments are required in the future.

Due to the requirements for the increase in the speed of the information processing and the decreases in the size and the weight, a further increase in the speed of rotation is required The eater the speed, the greater becomes the energy loss at the bearing.

As for the environment of the use of various information processing instruments, the use under severe environment is increasing. In particular, instruments mounted on automobiles such as automobile navigation instruments are required to be durable under conditions ranging from extreme cold to extreme heat when the environment of the use of automobiles is considered.

Therefore, for the lubricating oil for bearings used for instruments mounted on automobiles, it is required that the lubricating oil can be used at temperatures in the range as wide as from −40 to 80° C. without problems.

As the lubricating oil for fluid dynamic bearings, a lubricating oil exhibiting excellent viscosity characteristics such as excellent fluidity at low temperatures and a small decrease in the viscosity in a high temperature range, lower evaporability, excellent energy saving property (small consumption of electric power) and excellent heat stability in combination with the basic properties such as the lubricating property, the stability against degradation (the life), the property for preventing formation of sludge, the antiwear property and the anticorrosion property, is desired.

Heretofore, lubricating oils using a poly-α-olefin (PAO) or dioctyl sebacate (DOS) as the base oil have been used as the lubricating oil for fluid dynamic bearings. However, these lubricating oils cannot satisfy the requirements arising from the increase in the speed of information processing and the decreases in the size and the weight of the instruments, and lubricating oils using a polyol ester as the base oil are being used recently.

As the lubricating oil for fluid dynamic bearings using a polyol ester as the base oil, for example, (1) a lubricating oil for fluid dynamic bearings using a mixture of a polyol ester-based oil, which is obtained by the reaction of a polyhydric alcohol such as hexamethylene glycol, neopentyl glycol, decamethylene glycol, pentaerythritol and trimethylolpropane with a fatty acid having 5 to 20 carbon atoms such as heptanoic acid, octanoic acid, nonanoic acid and decanoic acid, and a diester-based oil such as dioctyl adipate and dioctyl sebacate as the base oil (for example, refer to Patent Reference 1) and a lubricating oil for fluid dynamic bearings using an ester of trimethylolpropane with a mixed acid comprising at least two monovalent fatty acids having 4 to 8 carbon atoms as the base oil (for example, refer to Patent Reference 2), are disclosed.

However, the above lubricating oils for fluid dynamic bearings using the polyol esters as the base oil have drawbacks in that the lubricating oils do not always sufficiently satisfy the above requirements for the properties and that the lubricating oils tend to be hydrolyzed and corrode metal materials due to the presence of the ester bond.

[Patent Reference 1] Japanese Patent Application Laid-Open No. 2001-279284

[Patent Reference 2] Japanese Patent Application Laid-Open No. 2004-91524

### DISCLOSURE OF THE INVENTION

Under the above circumstances, the present invention has an object of providing a lubricating oil composition for fluid dynamic bearings which exhibits excellent viscosity characteristics such as excellent fluidity at low temperatures and a small decrease in the viscosity in a high temperature range, lower evaporability, excellent energy saving property and excellent heat stability in combination with the basic properties such as the lubricating property, the stability against degradation, the property for preventing formation of sludge, the antiwear property and the anticorrosion property and is advantageously used for compact fluid dynamic bearings used under rotation at a high speed

As a result of intensive studies by the present inventors to achieve the above object to develop a lubricating oil composition for fluid dynamic bearings having excellent properties, it was found that the object could be achieved with a lubricating oil composition comprising at least 50% by mass of a specific ether compound and having a kinematic viscosity of a specific value or greater at 100° C.

The present invention has been completed based on such a knowledge.

The present invention provides:
(1) A lubricating oil composition for fluid dynamic bearings which comprises 50 to 100% by mass of an ether compound comprising at least one ether bond and having 11 to 34 carbon atoms as a base oil and has a kinematic viscosity of at least 2.2 mm²/s at 100° C.;
(2) A lubricating oil composition for fluid dynamic bearings described in (1), wherein the ether compound comprising at least one ether bond and having 11 to 34 carbon atoms has 1 to 4 ether bonds;
(3) A lubricating oil composition for fluid dynamic bearings described in (2), wherein the ether compound comprising at least one ether bond and having 11 to 34 carbon atoms is a monoether compound represented by a general formula (I):
   R¹—O—R² (I)
wherein R¹ represents a monovalent hydrocarbon group comprising a side chain and having 8 to 24 carbon atoms, and R² represents a monovalent hydrocarbon group having 3 to 10 carbon atoms;
(4) A lubricating oil composition for fluid dynamic bearings described in (2), wherein the ether compound comprising at least one ether bond and having 11 to 34 carbon atoms is a diether compound represented by a general formula (II):
   R³—O—R⁴—O—R⁵ (II)
wherein R³ and R⁵ each represent a monovalent hydrocarbon group, R⁴ represents a divalent hydrocarbon group, at least one of groups represented by R³, R⁴ and R⁵ has a side chain, and a number of carbon atoms in entire groups represented by R³, R⁴ and R⁵ is 11 to 34;
(5) A lubricating oil composition for fluid dynamic bearings described in (2), wherein the ether compound comprising at least one ether bond and having 11 to 34 carbon atoms is a tri- or tetraether compound represented by a general formula (III):
   R⁶—O—(R⁷—O)ₙ—R⁸ (III)
wherein R⁶ and R⁸ each represents a monovalent hydrocarbon group, R⁷ represents a divalent hydrocarbon group, represents an integer of 2 or 3, at least one of groups represented by R⁶, R⁷ and R⁸ has a side chain, and a number of carbon atoms in entire groups represented by R⁶, R⁷ and R⁸ is 11 to 34;
(6) A lubricating oil composition for fluid dynamic bearings described in any one of (1) to (5), which comprises at least one of phenol-based antioxidants and/or amine-based antioxidants;
(7) A lubricating oil composition for fluid dynamic bearings described in any one of (1) to (6), which comprises a lubricity improver additive;
(8) A lubricating oil composition for fluid dynamic bearings described in any one of (1) to (7), which comprises an electrically conductive additive;
(9) A lubricating oil composition for fluid dynamic bearings described in any one of (1) to (8), wherein an evaporation loss after a heat treatment at 120° C. for 24 hours is not more than 1.5% by mass;
(10) A lubricating oil composition for fluid dynamic bearings described in any one of (1) to (9) wherein a kinematic viscosity at a temperature of −20° C. is not more than 140 mm²/s, and a pour point is not more than −40° C.; and
(11) A lubricating oil composition for fluid dynamic bearings described in any one of (1) to (10), wherein a volume resistivity is not more than 1×10¹⁰ Ω·cm in substantial absence of particles of metals or particles of metal oxides.

In accordance with the present invention, a lubricating oil composition having excellent viscosity characteristics such as excellent fluidity at low temperatures and a small decrease in the viscosity in a high temperature range, exhibiting lower evaporability, excellent energy saving property and excellent heat stability and advantageously used for compact fluid dynamic bearings used under rotation at a high speed can be provided.

### THE MOST PREFERRED EMBODIMENT TO CARRY OUT THE INVENTION

It is necessary that the lubricating oil composition for fluid dynamic bearings of the present invention comprises 50 to 100% by mass of an ether compound comprising at least one ether bond and having 11 to 34 carbon atoms as the base oil.

As the ether compound, it has preferably 1 to 4 ether bonds. For example, the following compounds can be used.

A monoether compound represented by a general formula (I):
R¹—O—R² (I)
wherein R¹ represents a monovalent hydrocarbon group having 8 to 24 carbon atoms and a side chain, and R² represents a monovalent hydrocarbon group having 3 to 10 carbon atoms;

A diether compound represented by a general formula (II):
R³—O—R⁴—O—R⁵ (II)
wherein R³ and R⁵ each represent a monovalent hydrocarbon group, R⁴ represents a divalent hydrocarbon group, at least one of groups represented by R³, R⁴ and R⁶ has a side chain, and the number of carbon atom in the entire groups represented by R³, R⁴ and R⁵ is 11 to 34.

A tri- or tetraether compound represented by a general formula (III):
R⁶—O—(R⁷—O)ₙ—R⁸ (III)
wherein R⁶ and R⁸ each represent a monovalent hydrocarbon group, R⁷ represents a divalent hydrocarbon group, n represents an integer of 2 or 3, at least one of groups represented by R⁶, R⁷ and R⁸ has a side chain, and the number of carbon atom in the entire groups represented by R⁶, R⁷ and R⁸ is 11 to 34.

A tetraether compound is preferable in the compound represented by the general formula (III).

The monovalent hydrocarbon group comprising a side chain and having 8 to 24 carbon atoms which is represented by R¹ in the general formula (I) is a group having 8 to 24 carbon atoms in which at least one alkyl group alkenyl group, cycloalkyl group or aryl group is bonded to a linear alkyl group or alkenyl group as a side chain.

The position of bonding of the side chain is not particularly limited. The alkyl group or the alkenyl group may have a monovalent or divalent aromatic hydrocarbon group or a monovalent or divalent saturated or unsaturated alicyclic hydrocarbon group

Among these groups, a group in which at least one alkyl group is bonded to a linear alkyl group as the side chain is preferable

Specific examples of the group represented by R¹ include 2-ethylhexyl group, 2-propylheptyl group, 2-butyloctyl group, 2-pentylnonyl group, 2-hexyldecyl group, 2-heptylundecyl group, 2-octyldodecyl group, 2-nonyldodecyl group, 2-decyltetradecyl group, 3,5,5-trimethylhexyl group, 3,7-dimethyloctyl group and 3,3-diethylpentyl group.

Examples of the monovalent hydrocarbon group having 3 to 10 carbon atoms which is represented by R² include linear, branched and cyclic alkyl groups and alkenyl groups having 3 to 10 carbon atoms, aryl groups having 6 to 10 carbon atoms and aralkyl groups having 7 to 10 carbon atoms.

Specific examples of the group represented by R² include various types of propyl groups, various types of butyl groups, various types of pentyl groups, various types of hexyl groups, various types of heptyl groups, various types of octyl groups, various types of nonyl groups, various types of decyl groups, allyl group, propenyl group, various types of butenyl groups, various types of hexenyl groups, various types of octenyl groups, various types of decenyl groups, cyclopentyl group, cyclohexyl group, cyclopentenyl group, cyclohexenyl group, phenyl group, tolyl group, naphthyl group, benzyl group and phenetyl group.

Among these groups, alkyl groups such as various types of propyl groups, various types of butyl groups, various types of pentyl groups, various types of hexyl groups, various types of heptyl groups, various types of octyl groups, various types of nonyl groups and various types of decyl groups are preferable.

As the monoether compound represented by the general formula (I), monoether compounds having 18 to 32 carbon atoms are preferable, monoether compounds having 20 to 30 carbon atoms are more preferable and monoether compounds having 22 to 27 carbon atoms are most preferable.

Examples of the monoether compound include 2-propylheptyl decyl ether, 2-butyloctyl octyl ether, 2-butyloctyl decyl ether, 2-pentylnonyl hexyl ether, 2-pentylnonyl heptyl ether, 2-pentylnonyl octyl ether, 2-pentylnonyl nonyl ether, 2-pentylnonyl decyl ether, 2-hexyldecyl butyl ether, 2-hexyldecyl pentyl ether, 2-hexyldecyl hexyl ether, 2-hexyldecyl heptyl ether, 2-hexyldecyl octyl ether, 2-hexyldecyl decyl ether, 2-heptylundecyl propyl ether, 2-heptylundecyl butyl ether, 2-heptylundecyl pentyl ether, 2-heptylundecyl hexyl ether, 2-heptylundecyl heptyl ether, 2-heptylundecyl octyl ether, 2-heptylundecyl nonyl ether, 2-heptylundecyl decyl ether, 2-octyldodecyl propyl ether, 2-octyldodecyl butyl ether, 2-octyldodecyl pentyl ether, 2-octyldodecyl hexyl ether, 2-octyldodecyl heptyl ether, 2-octyldodecyl octyl ether, 2-octyldodecyl nonyl ether, 2-octyldodecyl decyl ether, 2-nonyltridecyl propyl ether, 2-nonyltridecyl butyl ether, 2-nonyltridecyl pentyl ether, 2-nonyltridecyl hexyl ether, 2-nonyltridecyl heptyl ether, 2-nonyltridecyl octyl ether, 2-decyltetradecyl propyl ether, 2-decyltetradecyl butyl ether, 2-decyltetradecyl pentyl ether, 2-decyltetradecyl hexyl ether and 3,7-dimethyloctyl decyl ether.

The monoether compound represented by the general formula (I) can be prepared in accordance with a conventional process such as a process shown by the following reaction scheme (A): 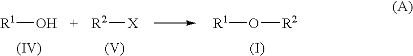
wherein X represents a halogen atom, and R¹ and R² are as defined above.

As shown above, the monoether compound represented by the general formula (I) can be obtained by the reaction of a monovalent hydrocarbyl alcohol (IV) comprising a side chain and having 8 to 24 carbon atoms with a halogenated hydrocarbon (V) having 3 to 10 carbon atoms in the presence of a hydrogen halide scavenger such as sodium hydroxide.

The hydrocarbyl alcohol comprising a side chain and having 8 to 24 carbon atoms which is represented by the general formula (IV) can be prepared in accordance with a conventional process such as the process using the Guerbet reaction in which two molecules of a primary alcohol are condensed under a condition of a high temperature and a high pressure, the oxo process or the process using oligomerization of an α-olefin into a dimer or greater oligomers.

In the general formula (II), it is necessary that at least one of the monovalent hydrocarbon groups represented by R³ and R⁵ and the divalent hydrocarbon group represented by R⁴ has a side chain, and the number of carbon atoms in the entire groups represented by R³, R⁴ and R⁵ is 11 to 34.

Examples of the monovalent hydrocarbon group represented by R³ or R⁵ include linear alkyl and alkenyl groups and linear alkyl and alkenyl groups to which at least one alkyl, alkenyl, cycloalkyl or aryl group is bonded as the side chain.

When the above group has a side chain, the position of bonding of the side chain is not particularly limited.

The alkyl group or the alkenyl group may have a monovalent or divalent aromatic hydrocarbon group or a monovalent or divalent saturated or unsaturated alicyclic hydrocarbon group.

Among the above groups, linear alkyl groups and groups having 6 to 12 carbon atoms in which at least one alkyl group is bonded to a linear alkyl group as the side chain are preferable.

R³ and R⁵ may represent the same groups or different groups. It is preferable that R³ and R⁵ represent the same groups from the standpoint of the easiness of production of the diether compound represented by the general formula (II).

Examples of the groups represented by R³ and R⁵ include octyl group, nonyl group, decyl group, dodecyl group, 2-ethylhexyl group, 2-propylheptyl group, 2-butyloctyl group, 3,5,5-trimethylhexyl group and 3,7-dimethyloctyl group.

Examples of the divalent hydrocarbon group represented by R⁴ in the general formula (II) include linear alkylene and alkenylene groups and linear alkylene and alkenylene groups to which at least one alkyl, alkenyl, cycloalkyl or aryl group is bonded as the side chain.

The alkylene and alkenylene group described above may have a divalent aromatic hydrocarbon group or a divalent saturated or unsaturated alicyclic hydrocarbon group.

Among the above groups, linear alkylene groups and groups having 2 to 10 carbon atoms in which at least one alkyl group is bonded to a linear alkylene group as the side chain are preferable.

Examples of the group represented by R⁴ include ethylene group, trimethylene group, tetramethylene group, pentamethylene group, hexamethylene group, heptamethylene group, octamethylene group, decamethylene group, propylene group, 3-methylpentylene group, 3,3-diethylpentylene group and 2,2-dimethyltrimethylene group.

As the diether compound represented by the general formula (II), diether compounds having 18 to 32 carbon atoms are preferable, diether compounds having 20 to 30 carbon atoms are more preferable, and diether compounds having 22 to 27 carbon atoms are most preferable.

Examples of the diether compound include 1,4-bis(2-ethylhexoxy)butane, 1,6-bis(2-ethylhexoxy)hexane, 1,8-bis(2-ethylhexoxy)octane, 1,10-bis(2-ethylhexoxy)decane, 1,2-bis(2-propylheptoxy)ethane, 1,3-bis-(2-propylheptoxy)propane, 1,4-bis(2-propylheptoxy)butane, 1,6-bis-(2-propylheptoxy)hexane, 1,8-bis(2-propylheptoxy)octane, 1,10-bis-(2-propylheptoxy)decane, 1,2-bis(2-butyloctoxy)ethane, 1,3-bis(2-butyl-octoxy)propane, 1,4-bis(2-butyloctoxy)butane, 1,6-bis(2-butyloctoxy)hexane, 1,8-bis(2-butyloctoxy)octane, 1,2-bis(3,5,5-trimethylhexoxy)ethane, 1,3 -bis(3,5,5-trimethylhexoxy)propane, 1,4-bis(3,5,5-trimethylhexoxy)butane, 1,6-bis(3,5-trimethylhexoxy)hexane, 1,8-bis(3,5,5-trimethylhexoxy)octane, 1,10-bis(3,5,5-trimethylhexoxy)decane, 1,5-bis(heptoxy)-3-methylpentane, 1,5-bis(octoxy)-3-methylpentane, 1,5-bis(nonyloxy)-3-methylpentane, 1,5-bis(decyloxy)-3-methylpentane, 1,5-bis(dodecyloxy)-3-methylpentane, 1,3-bis(octoxy)-2,2-dimethylpropane, 1,3-bis(nonyloxy)-2,2-dimethylpropane, 1,3-bis(decyloxy)-2,2-dimethylpropane, 1,3-bis-(dodecyloxy)-2,2-dimethylpropane, 1,5-bis(hexoxy)-3,3-diethylpentane, 1,5-bis(heptoxy)-3,3-diethylpentane, 1,5-bis(octoxy)-3,3-diethylpentane and 1,5-bis(decyloxy)-3,3-diethylpentane.

The diether compound represented by the general formula (II) can be prepared in accordance with a conventional process, for example, when R³ and R⁴ represent the same group, in accordance with the process shown by the following reaction scheme (B): 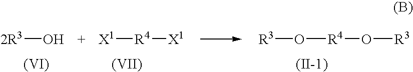
wherein X¹ represents a halogen atom, and R³ and R⁴ are as defined above.

As shown above, the diether compound represented by the general formula (II-1) can be obtained by the reaction of a monohydric hydrocarbyl alcohol (VI) and a dihalogenated hydrocarbon (VII) in the presence of a hydrogen halide scavenger such as sodium hydroxide.

Among the hydrocarbyl alcohol represented by the general formula (VI), hydrocarbyl alcohols having a side chain can be produced in accordance with a conventional process such as the process using the Guerbet reaction in which two molecules of a primary alcohol are condensed under a condition of a high temperature and a high pressure, the oxo process or the process using oligomerization of an α-olefin into a dimer or greater oligomers.

In the general formula (III), it is necessary that at least one of the monovalent hydrocarbon groups represented by R⁶ and R⁸ and the divalent hydrocarbon group represented by R⁸ has a side chain, and the number of carbon atoms in the entire groups represented by R⁶, R⁷ and R⁸ is 11 to 34.

Examples of the monovalent hydrocarbon group represented by R⁶ or R⁸ include linear alkyl and alkenyl groups and linear alkyl and alkenyl groups to which at least one alkyl, alkenyl, cycloalkyl or aryl group is bonded as the side chain.

When the above group has a side chain, the position of bonding of the side chain is not particularly limited.

The alkyl group or the alkenyl group may have a monovalent or divalent aromatic hydrocarbon group or a monovalent or divalent saturated or unsaturated alicyclic hydrocarbon group.

Among the above groups, linear alkyl groups and groups having 6 to 12 carbon atoms in which at least one alkyl group is bonded to a linear alkyl group as the side chain are preferable.

R⁶ and R⁸ may represent the same groups or different groups. It is preferable that R⁶ and R⁸ represent the same groups from the standpoint of the easiness of production of the tri- or tetraether compound represented by the general formula (III).

Examples of the groups represented by R⁶ and R⁸ include octyl group, nonyl group, decyl group, dodecyl group, 2-ethylhexyl group, 2-propylheptyl group, 2-butyloctyl group, 3,5,5-trimethylhexyl group and 3,7-dimethyloctyl group.

Examples of the divalent hydrocarbon group represented by R⁷ in general formula (III) include linear alkylene and alkenylene groups and linear alkylene and alkenylene groups to which at least one alkyl, alkenyl cycloalkyl or aryl group is bonded as the side chain.

The alkylene and alkenylene group described above may have a divalent aromatic hydrocarbon group or a divalent saturated or unsaturated alicyclic hydrocarbon group.

Among the above groups, linear alkylene groups and groups having 2 to 10 carbon atoms in which at least one alkyl group is bonded to a linear alkylene group as the side chain are preferable.

Examples of the group represented by R⁷ include ethylene group, trimethylene group, 2-methylethylene group, tetramethylene group, pentamethylene group, hexamethylene group, heptamethylene group, octamethylene group, decamethylene group, propylene group, 3-methylpentylene group, 3,3-diethylpentylene group and 2,2-dimethyl-trimethylene group.

As the tri- and tetraether compounds represented by the general formula (III), compounds having 18 to 32 carbon atoms are preferable, compounds having 20 to 30 carbon atoms are more preferable, and compounds having 22 to 27 carbon atoms are most preferable.

Examples of the tri- and tetraether compounds include di(ethylene glycol) di-2-ethylhexyl ether, di(ethylene glycol) di-3,5,5-trimethylhexyl ether, di(ethylene glycol) di-2-propylheptyl ether, di(ethylene glycol) di-2-butyloctyl ether, di(1,3-propylene glycol) di-2-ethylhexyl ether, di(1,3-propylene glycol) di-3,5,5-trimethylhexyl ether, di(1,3-propylene glycol) di-2-propylheptyl ether, di(1,3-propylene glycol) di-2-butyloctyl ether, di(1,2-propylene glycol) di-2-ethylhexyl ether, di(2-propylene glycol) dinonyl ether, di(1,2-propylene glycol) di-3,5,6-trimethylhexyl ether, di(1,2-propylene glycol) di-2-propylheptyl ether, di(1,2-propylene glycol) didecyl ether, di(1,2-propylene glycol) di-2-butyloctyl ether, di(1,4-butanediol) di-2-ethylhexyl ether, di(1,4-butanediol) di-3,5,5-trimethylhexyl ether, di(1,5-butanediol) di-2-ethylhexyl ether, triethylene glycol) di-2-ethylhexyl ether, tri(ethylene glycol) di-3,5,5-trimethylhexyl ether, tri(ethylene glycol) di-2-propylheptyl ether, tri(ethylene glycol) di-2-butyloctyl ether, tri(1,3-propylene glycol) di-2-ethylhexyl ether, tri(1,3-propylene glycol) di-3,5,5-trimethylhexyl ether, tri(1,3-propylene glycol) di-2-propylheptyl ether, tri(1,2-propylene glycol) di-2-ethylhexyl ether, tri(1,2-propylene glycol) dinonyl ether, tri(1,2-propylene glycol) di-3,5,5-trimethylhexyl ether, tri(1,2-propylene glycol) di-2-propylheptyl ether, tri(1,2-propylene glycol) didedyl ether, tri(1,4butanediol) di-2-ethylhexyl ether and tri(1,4-butanediol) di-3,5,6-trimethylhexyl ether.

The tri- or tetraether compound represented by the general formula (III) can be prepared in accordance with a conventional process, for example, when R⁶ and R⁸ represent the same group, in accordance with the process shown by the following reaction scheme (C): 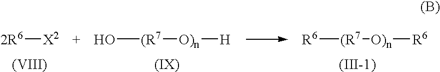
wherein X² represents a halogen atom, and R⁶, R⁷ and n are as defined above.

As shown above, the tri- or tetraether compound represented by the general formula (III-1) can be obtained by the reaction of a diol (IX) and a monohalogenated hydrocarbon (VIII) in the presence of a hydrogen halide scavenger such as sodium hydroxide.

Among the above diols represented by the general formula (IX), diols having a side chain can be produced in accordance with a conventional process such as dimerization of propylene oxide.

In the lubricating oil composition of the present invention, it is preferable that at least one compound is suitably selected from the monoether compound, the diether compound, the triether compound and the tetraether compound described above and used as the base oil so that the prescribed properties required for the lubricating oil composition which are described below are satisfied

The content of the entire ether compounds is selected in the range of 50 to 100% by mass.

When the content of the entire ether compounds selected from the monoether compound, the diether compound, the triether compound and the tetraether compound is at least 50%, a lubricating oil composition satisfying the requirements as the lubricating oil composition for fluid dynamic bearings can be obtained.

It is preferable that the content is 70 to 100% by mass, more preferably 50 to 100% by mass and most preferably 90 to 100% by mass.

Where desired, the lubricating oil composition of the present invention may comprise other base oils in an amount of not more than 50% by mass, preferably not more than 30% by mass, more preferably not more than 20% by mass and most preferably not more than 10% by mass as long as the effect of the present invention is not adversely affected.

Examples of the other base oil include mineral oils, poly-α-olefin oils and ester-based oils such as diesters and polyol esters.

The lubricating oil composition of the present invention has a kinematic viscosity of at least 2.2 mm²/s at the temperature of 100° C.

When the kinematic viscosity is at least 2.2 mm²/s, excellent rigidity as the bearing is exhibited in the use at high temperatures, and a rotating sleeve member can be supported sufficiently to exhibit the excellent durability.

The maximum kinematic viscosity at 100° C. is, in general, about 3.5 mm²/s.

It is preferable that the kinematic viscosity at −20° C. is not more than 140 mm²/s.

When the kinematic viscosity is not more than 140 mm²/s at −20° C., the lubricating property can be sufficiently exhibited under the environment of low temperatures.

It is preferable that the lubricating oil composition of the present invention has a viscosity index of at least 100.

When the viscosity index is at least 100, the change of the viscosity with temperature is small, and the lubricating oil composition works well in the high temperature range and in the low temperature range.

It is more preferable that the viscosity index is at least 120 and most preferably at least 125.

The kinematic viscosity and the viscosity index are obtained in accordance with the method of Japanese Industrial Standard K2283.

It is preferable that the pour point obtained by the measurement in accordance with the method of Japanese Industrial Standard K2265 is not more than −40° C. and more preferably not more than −45° C.

When the pour point is not more than −40° C., the lubricating oil composition works well in the low temperature range.

It is preferable that the lubricating oil composition of the present invention has an evaporation loss after a heat treatment at 120° C. for 24 hours is not more than 1.5% by mass.

When the evaporation loss is not more than 1.5% by mass, the lubricating property can be exhibited for a long time with stability.

It is more preferable that the evaporation loss is not more than 1.0% by mass

The evaporation loss is obtained by the measurement in accordance with the method for the test of heat stability of Japanese Industrial Standard C201.

Where desired, the lubricating oil composition of the present invention may comprise various additives such as antioxidants, lubricity impover additives, electrically conductive additives, rust preventives, metal deactivators, defoaming agents and viscosity index improvers as long as the effect of the present invention is not adversely affected.

Examples of the antioxidant include amine-based antioxidants, phenol-based antioxidants and sulfur-based antioxidants.

Examples of the amine-based antioxidant include monoalkyl-diphenylamine-based antioxidants such as monooctyldiphenylamine and monononyldiphenylamine; dialkyldiphenylamine-based antioxidants such as 4,4′-dibutyldiphenylamine, 4,4′-dipentyldiphenylamine, 4,4′-dihexyldiphenylamine, 4,4′-diheptyldiphenylamine, 4,4′-dioctyldiphenylamine and 4,4′-dinonyldiphenylamine; polyalyldiphenylamine-based anti-oxidants such as tetrabutyldiphenylamine, tetrahexyldiphenylamine, tetraoctyldiphenylamine and tetranonyldiphenylamine; and naphthylamine-based antioxidants such as α-naphthylamine, phenyl-α-naphthylamine, butylphenyl-α-naphthylamine pentylphenyl-α-naphthylamine, hexylphenyl-α-naphthylanmine, heptylphenyl-α-naphthylamine octylphenyl-α-naphthylamine and nonylphenyl-α-naphthylamine. Among these amine-based antioxidants dialkyldiphenylamine-based antioxidants are preferable.

Examples of the phenol-based antioxidants include monophenol-based antioxidants such as 2,6-di-tert-butyl-4-methylphenol, 2,6-di-tert-butyl-4-ethylphenol and octadecyl 3-(3,5-di-tert-butyl-4-hydroxyphenyl)propionate; and bisphenol-based antioxidants such as 4,4′-methylene-bis(2,6-di-tert-butylphenol) and 2,2′-methylenebis(4-ethyl-6-tert-butylphenol).

Examples of the sulfur-based antioxidant include phenothiazine, pentaerythritol tetrakis(3-laurylthiopropionate), bis(3,5-di-tert-butyl-4-hydroxybenzyl) sulfide, thiodiethylene bis(3-(3,5-di-tert-butyl-4-hydroxyphenyl)propionate and 2,6-di-tert-butyl-4-(4,6-bis(octylthio)1,3,5-triazime-2-methylamino)phenol.

The antioxidant described above may be used singly or in combination of two or more Among these antioxidant phenol-based and/or amine-based antioxidants are preferable.

It is preferable that the amount of the antioxidant is in the range of 0.01 to 10% by mass and more preferably in the range of 0.03 to 5% by mass based on the amount of the entire composition.

As the lubricity impover additive, oiliness agents and friction modifiers can be used.

Examples of the oiliness agent include saturated and unsaturated aliphatic monocarboxylic acids such as stearic acid and oleic acid, polymerized fatty acids such as dimer acid and hydrogenated dimer acid, hydroxyfatty acids such as ricinoleic acid and 12-hydroxystearic acid, saturated and unsaturated aliphatic mono alcohols such as lauryl alcohol and oleyl alcohol, saturated and unsaturated aliphatic mono amines such as stearylamine and oleylamine and saturated and unsaturated aliphatic monocarboxylic acid amides such as lauramide and oleamide.

It is preferable that the amount of the oiliness agent is in the range of 0.01 to 10% by mass and more preferably in the range of 0.1 to 5% by mass based on the total amount of the entire composition.

Examples of the friction modifier include agents conventionally used as the extreme pressure agent. In particular, phosphoric acid esters, amine salts of phosphoric acid esters and sulfur-based extreme pressure agents can be used.

Examples of the phosphoric acid ester include phosphoric acid esters, acidic phosphoric acid esters, phosphorous acid esters and acidic phosphorous acid esters represented by the following general formulae (X) to (XIV):

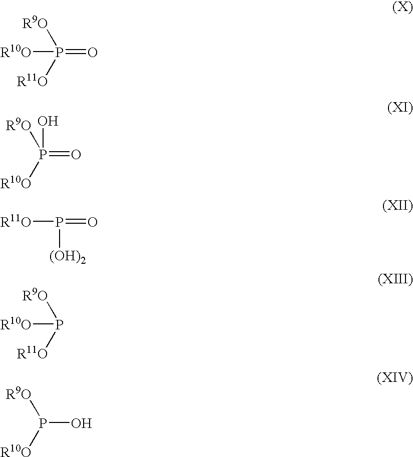

In the above general formulae (X) to (XIV), R⁹ to R¹¹ each represent an alkyl group or alkenyl group having 4 to 30 carbon atoms, an alkylaryl group or an arylalkyl group and may represent the same group or different groups.

Examples of the phosphoric acid ester include triaryl phosphates, trialkyl phosphates, trialkylaryl phosphates, triarylalkyl phosphates and trialkenyl phosphates. Specific examples include triphenyl phosphate, tricresyl phosphate, benzyl diphenyl phosphate, ethyl diphenyl phosphate, tributyl phosphate, ethyl dibutyl phosphate, cresyl diphenyl phosphate, dicresyl phenyl phosphate, ethylphenyl diphenyl phosphate, diethylphenyl phenyl phosphate, propylphenyl diphenyl phosphate, dipropylphenyl phenyl phosphate, triethylphenyl phosphate, tripropylphenyl phosphate, butylphenyl diphenyl phosphate, dibutylphenyl phenyl phosphate, tributylphenyl phosphate, trihexyl phosphate, tri(2-ethylhexyl) phosphate, tridecyl phosphate, trilauryl phosphate, trimyristyl phosphate, tripalmityl phosphate, tristearyl phosphate and trioleyl phosphate.

Examples of the acidic phosphoric acid ester include 2-ethylhexyl acid phosphate, ethyl acid phosphate, butyl acid phosphate, oleyl acid phosphate, tetracosyl acid phosphate, isodecyl acid phosphate, lauryl acid phosphate, tridecyl acid phosphate, stearyl acid phosphate and isostearyl acid phosphate.

Examples of the phosphorous acid ester include triethyl phosphite, tributyl phosphite, triphenyl phosphite, tricresyl phosphite, tri(nonylphenyl) phosphite, tri(2-ethylhexyl) phosphite, tridecyl phosphite, trilauryl phosphite triisooctyl phosphite, diphenyl isodecyl phosphite, tristearyl phosphite and trioleyl phosphite.

Examples of the acidic phosphorous acid ester include dibutyl hydrogenphosphite, dilauryl hydrogenphosphite, dioleyl hydrogen-phosphite, distearyl hydrogenphosphite and diphenyl hydrogenphosphite. Among the above phosphoric esters tricresyl phosphite and triphenyl phosphate are preferable.

Examples of the amine forming an amine salt with the above compounds include monosubstituted amines, disubstituted amines and trisubstituted amines represented by the following general formula (XV):

R¹²ₚ—NH₂₋ₚ (XV)

wherein R¹² represents an alkyl group or alkenyl group having 3 to 30 carbon atoms, an aryl group or arylalkyl group having 6 to 30 carbon atoms or a hydroxyalkyl group having 2 to 30 carbon atoms and n represents 1, 2 or 3. When a plurality of R¹² are present, the plurality of R² may represent the same groups or different groups. The alkyl group or alkenyl group having 3 to 30 carbon atoms which is represented by R¹² in the above general formula (XV) may be any of a linear group, a branched group and a cyclic group.

Examples of the monosubstituted amine include butylamine, pentylamine, hexylamine, cyclohexylamine, octylamine, laurylamine, stearylamine, oleylamine and benzylamine. Examples of the disubstituted amine include dibutylamine, dipentylamine, dihexylamine, dicyclohexylamine, dioctylamine, dilaurylamine, distearylamine, dioleylamine, dibenzylamine, stearyl-monoethanolamine, decyl-monoethanolamine, hexyl-monopropanolamine, benzyl-monoethanolamine, phenyl-monoethanolamine and tolyl-monopropanolamine. Examples of the trisubstituted amine include tributylamine, tripentylamine, trihexylamine, tricyclohexylamine, trioctylamine, trilaurylamine, tristearylamine, trioleylamine, tribenzylamine, dioleyl-monoethanolamine, dilauryl-monopropanolamine, dioctyl-monoethanolamine, dihexyl-monopropanolamine, dibutyl-monopropanolamine, oleyl-diethanolamine, stearyl-dipropanolamine, lauryl-diethanolamine, octyl-dipropanolamine, butyl-diethanolamine, benzyl-diethanolamine, phenyl-diethanolamine, tolyl-dipropanolamine, xylyl-diethanolamine, triethanolamine and tripropanolamine.

As the sulfur-based extreme pressure agent, any agent which has sulfur atom in the molecule, is dissolved or dispersed uniformly in the base oil of the lubricating oil and can exhibit the extreme pressure property and the excellent friction property can be used.

Examples of the sulfur-based extreme pressure agent include sulfurized oils and fats, sulfurized fatty acids, sulfurized esters, sulfurized olefins dihydrocarbyl polysulfides, thiadiazole compounds, thiophosphoric esters (thiophosphites and thiophosphates), alkylthiocarbamoyl compounds thiocarbamate compounds, thioterpene compounds and dialkylthiodipropionate compounds.

The sulfurized oil and fat is obtained by bringing oil and fat such as lard, whale oil, plant oils and fish oils into reaction with sulfur or a compound having sulfur. The content of sulfur is not particularly limited. In general a content in the range of 5 to 30% by mass is preferable.

Examples of the sulfurized oil and fat include sulfurized lard sulfurized rapeseed oil, sulfurized castor oil, sulfurized soybean oil and sulfurized rice bran oil.

Examples of the sulfurized fatty acid include sulfurized oleic acid. Examples of the sulfurized ester include sulfurized methyl oleate and sulfurized octyl esters of fatty acids derived from rice bran.

Examples of the olefin sulfide include compounds represented by the following general formula (XVI):
R¹³—S_{q}—R¹⁴ (XVI)
wherein R¹³ represents an alkenyl group having 2 to 15 carbon atoms, R¹⁴ represents an alkyl group or alkenyl group having 2 to 15 carbon atoms, and q represents an integer of 1 to 8.

The above compounds can be obtained by bringing an olefin having 2 to 15 carbon atoms or a dimer, a trimer or a tetramer thereof into reaction with a sulfurizing agent such as sulfur and sulfur chloride. As the olefin, propylene, isobutene and diisobutene are preferable.

The dihydrocarbyl polysulfide is a compound represented by the following general formula (XVII):
R¹⁵—Sᵣ—R¹⁶ (XVII)
wherein R¹⁵ and R¹⁶ each represent an alkyl group or cyclic alkyl group having 1 to 20 carbon atoms, an aryl group having 6 to 20 carbon atoms, an alkylaryl group having 7 to 20 carbon atoms or an arylalkyl group having 7 to 20 carbon atoms and may represent the same group or different groups, and r represents an integer of 1 to 8. When R¹⁵ and R¹⁶ represent alkyl groups, the compound is a compound called an alkyl sulfide.

Examples of the group represented by R¹⁵ and R¹⁶ in general formula (XVII) include methyl group, ethyl group, n-propyl group, isopropyl group, n-butyl group, isobutyl group, sec-butyl group, tert-butyl group, various types of pentyl groups, various types of hexyl groups, various types of heptyl groups, various types of octyl groups, various types of nonyl groups, various types of decyl groups, various types of dodecyl groups, cyclohexyl group, cyclooctyl group, phenyl group, naphthyl group, tolyl group, xylyl group, benzyl group and phenetyl group.

Examples of the dihydrocarbyl polysulfide include dibenzyl polysulfide, various types of dinonyl polysulfides, various types of didodecyl polysulfides, various types of dibutyl polysulfides, various types of dioctyl polysulfides, diphenyl polysulfide and dicyclohexyl polysulfide.

As the thiadiazole compound, for example, 1,3,4-thiadiazoles, 1,2,4-thiadiazole compounds and 1,4,5-thiadiazoles which are represented by the following general formulae (XVIII) are preferable. 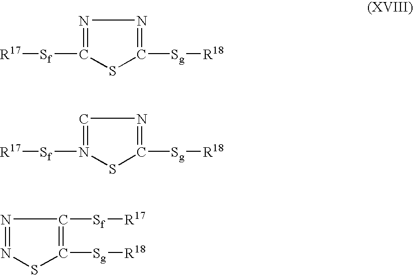
In the above formulae, R¹⁷ and R¹⁸ each represent hydrogen atom or a hydrocarbon group having 1 to 20 carbon atoms, and f and g each represent an integer of 0 to 8.

Specific examples of the thiadiazole compound include 2,5-bis(n-hexyldithio)-1,3,4-thiadiazole, 2,5-bis(n-octyldithio)-1,3,4-thiadiazole, 2,5-bis(n-nonyldithio)-1,3,4-thiadiazole, 2,5-bis(1,1,3,3-tetramethylbutyldithio-1,3,4-thiadiazole, 3,5-bis(n-hexyldithio)-1,2,4-thiadiazole, 3,5-bis(n-octyldithio)-1,2,4-thiadiazole, 3,5-bis(n-nonyldithio)-1,2,4-thiadiazole, 3,5-bis(1,1,3,3-tetramethylbutyldithio)-1,2,4-thiadiazole, 4,5-bis(n-hexyldithio)-1,2,3-thiadiazole, 4,6-bis(n-octyldithio)-1,2,3-thiadiazole, 4,5-bis(n-nonyldithio)-1,2,3-thiadiazole and 4,5-bis(1,1,3,3-tetramethylbutyldithio)-1,2,3-thiadiazole.

Examples of the thiophosphoric acid ester include alkyl trithiophosphites, aryl and alkylaryl thiophosphates and zinc dilauryl-dithiophosphate. Lauryl trithiophosphite and triphenyl thiophosphate are preferable.

Examples of the alkylthiocarbamoyl compound include compounds represented by the following general formula (XIX): 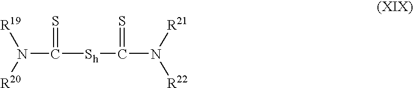
wherein R¹⁹ to R²² each represent an alky group having 1 to 20 carbon atoms, and h represents an integer of 1 to 8.

Examples of the alkylthiocarbamoyl compound include bis(dimethylthiocarbamoyl) monosulfide, bis(dibutylthiocarbamoyl) monosulfide, bis(dimethylthiocarbamoyl) disulfide, bis(dibutylthiocarbamoyl) disulfide, bis(diamylthiocarbamoyl) disulfide and bis(dioctylthiocarbamoyl) disulfide.

Examples of the thiocarbamate compound include zinc dialkyldithiocarbamates. Examples of the thioterpene compound include reaction products of phosphorus pentaoxide and pinene. Examples of the dialkyl thiodipropionate compound include dilauryl thiodipropionate and distearyl thiodipropionate.

Among these compounds, thiadiazole compounds and benzyl sulfide are preferable from the standpoint of the property as the extreme pressure agent, the friction property and stability against oxidation and heat.

It is preferable that the amount of the friction modifier is in the range of 0.01 to 10% by mass and more preferably 0.05 to 5% by mass based on the total amount of the entire composition.

It is preferable that the lubricating oil composition of the present invention has a volume resistivity of not more than 1×10¹⁰ Ω·cm in the substantial absence of particles of metals or particles of metal oxides.

When the volume resistivity is not more than 1×10¹⁰ Ω·cm, the lubricating oil composition exhibits the excellent antistatic property,

The minimum value of the volume resistivity is not particularly limited. In general, the minimum value is about 1×10⁷ Ω·cm.

The above values of the volume resistivity are obtained in accordance with the method of Japanese Industrial Standard C2102.

The lubricating oil composition of the present invention may comprise an electrically conductive additive so that the volume resistivity is adjusted at not more than 1×10¹⁰ Ω·cm.

As the electrically conductive additive, non-metallic antistatic agents such as amine derivatives, succinic acid derivatives, poly(oxyalkylene) glycols and partial esters of polyhydric alcohols are preferable. It is preferable that the amount of the electrically conductive additive is 0.01 to 10% by mass based on the total amount of the entire composition.

Examples of the amine derivative include polyloxyethylene)alkylamines represented by the following general formula: 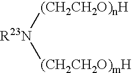
wherein R²³ represents an alkyl group having 1 to 18 carbon atoms; poly(oxyethylene)alkylamides represented by the following general formula: 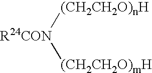
wherein R²⁴ represents an alkyl group having 1 to 18 carbon atoms; and condensation products of a polyethyleneimine such as tetraethylenepentamine (TEPE) and a fatty acid. The condensation product of TEPE and stearic acid is preferable.

Examples of the succinic acid derivative include polybutenylsuccinimide.

As the poly(oxyalkylene) glycol, compounds represented by the following general formula (XX):
R²⁵—O—(R²⁶—O)_{d}—(R²⁷—O)ₑ—(R²⁸—O)_{f}—R²⁹ (XX)
or mixtures of the above compounds are preferable.

In the general formula (XX), R²⁵ and R²⁹ each independently represents hydrogen atom, an alkyl group having 1 to 24 carbon atoms, phenyl group or an alkylaryl group having 7 to 24 carbon atoms, R²⁶, R²⁷ and R²⁸ each independently represents an alkylene group having 2 to 18 carbon atoms, d, e and f each independently represent a number of 0 to 50, and the total of the numbers represented by d to f is 9 to 50. (R²⁶—O), (R²⁷—O) and (R²⁸—O) represent constituting units which may be the same with or different from each other.

Among these compounds, poly(oxyethylene) alkyl ethers represented by the following general formula
R³⁰O(CH₂CH₂O)ₙH
wherein R³⁰ represents an alkyl group having 1 to 18 carbon atoms, and n represents a number of 1 to 10;

poly(oxyethylene) alkylphenyl ethers represented by the following general formula:
R³¹—Q—O(CH₂CH₂O)ₙH
wherein R³¹ represents an alkyl group having 1 to 18 carbon atoms, Q represents an aromatic residue group, and n represents a number of 1 to 10; and

poly(oxyethylene) glycol fatty acid esters represented by the following general formula:
R³²COO(CH₂CH₂O)ₙH
wherein R³² represents an alkyl group having 1 to 18 carbon atoms, and n represents a number of 1 to 10; are preferable.

Examples of the partial ester of a polyhydric alcohol include fatty acid esters of sorbitan represented by the following general formula: 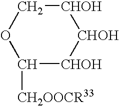
wherein R³³ represents an alkyl group having 1 to 18 carbon atoms, and n and m each represents a number of 1 to 10, such as sorbitan monooleate and sorbitan dioleate;

fatty acid esters of glycerol represented by the following general formula: 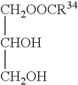
wherein R³⁴ represents an alkyl group having 1 to 18 carbon atoms, and n and m each represent a number of 1 to 10, such as glycerol monooleate and glycerol dioleate; and

partial ester compounds of fatty acids having 1 to 24 carbon atoms with polyhydric alcohols such as neopentyl glycol, trimethylolpropane and pentaerythritol.

Examples of the rust preventive include alkyl or alkenylsuccinic acid derivatives such as the half ester of dodecenylsuccinic acid, octadecenylsuccinic acid anhydride and dodecenylsuccinic acid amide; partial esters of polyhydric alcohols such as sorbitan monooleate, glycerol monooleate and pentaerythritol monooleate, amines such as rosin amine and N-oleylsarcosine and amine salts of dialkylphosphites.

It is preferable that the amount of the rust preventive is in the range of 0.01 to 5% by mass and more preferably 0.05 to 2% by mass based on the amount of the entire composition.

Examples of the metal deactivator include benzotriazole-based compounds, thiadiazole-based compounds and gallic acid ester-based compounds.

It is preferable that the amount of the metal deactivator is in the range of 0.01 to 0.4% by mass and more preferably 0.01 to 0.2% by mass based on the total amount of the entire composition.

As the defoaming agent, for example, liquid silicones are preferable. For example, methylsilicones, fluorosilicones and polyacrylates can be used.

It is preferable that the amount of the defoaming agent is in the range of 0.0005 to 0.01% by mass based on the total amount of the entire composition.

Examples of the viscosity index improver include polyalkyl methacrylates, polyalkylstyrenes, polybutenes and olefin copolymers such as ethylene-propylene copolymers, styrene-diene copolymers and styrene-maleic anhydride esters.

It is preferable that the amount of the viscosity improver is in the range of 0.1 to 15% by mass and more preferably in the range of 0.5 to 7% by mass based on the total amount of the entire composition.

The lubricating oil composition for fluid dynamic bearings of the present invention can be advantageously used for fluid dynamic bearings used in rotating devices for driving magnetic disks and optical disks such as FD, MO, minidisks, compact disks, DVD and hard disks.

### EXAMPLES

The present invention will be described more specifically with reference to examples in the following. However, the present invention is not limited to the examples.

The properties of a lubricating oil composition were measured in accordance with the following methods.

(1) Kinematic Viscosity

The kinematic viscosity was measured in accordance with the method of Japanese Industrial Standard K2283 at −100° C. and −20° C.

(2) Viscosity Index

The viscosity index was measured in accordance with the method of Japanese Industrial Standard K2283.

(3) Pour Point

The pour point was measured in accordance with the method of Japanese Industrial Standard K2269.

(4) Evaporation Loss

In accordance with the method for the test of heat stability of Japanese Industrial Standard C201, the evaporation loss in the heat treatment at 120° C. for 24 hours was measured.

(5) Test of Hydrolysis

Into a glass bottle, 75 g of a sample oil, 25 g of distilled water and a weighed amount of a copper catalyst were placed, and the bottle was tightly closed. After the bottle was rotated for 48 hours in a vessel kept at the constant temperature of 93° C., the decrease in the mass of the copper catalyst and the increase in the total acid value of the oil layer were measured.

The smaller the decrease in the mass of the copper catalyst and the smaller the increase in the total acid value of the oil layer, the more excellent the property of hydrolysis.

As the copper catalyst, a catalyst of a material C1100P specified in Japanese Industrial Standard H3100 and having a size of 13×51×1 mm was used. In the rotation of the glass bottle, the glass bottle was rotated at a speed of 5 rpm while the direction of rotation was upside-down during every revolution.

(6) Volume Resistivity

The volume resistivity was measured in accordance with the method of Japanese Industrial Standard C201.

### Preparation Example 1

Into a 2 liter glass flask, 300 g of 2-hexyldecanol, 300 g of 1-bromodecane, 30 g of tetrabutylammonium bromide and 500 g of a 30% by mass aqueous solution of sodium hydroxide were placed, and the reaction was allowed to proceed at 50° C. for 20 hours under stirring.

After the reaction was completed, the reaction mixture was transferred to a separatory funnel, and the aqueous phase was removed by filtration. The remaining organic phase was washed with 500 ml of water 5 times.

From the organic phase, 2-hexyldecyl decyl ether was separated by distillation under a reduced pressure.

In accordance with similar reactions using a branched eicosanol which was obtained by dimerization of decene and 2-hexyldecanol and 2-octyldodecanol which were obtained in accordance with the Guerbet reaction, a branched eicosyl butyl ether, 2-hexyldecyl nonyl ether, 2-octyldodecyl pentyl ether and 2-octyldodecyl hexyl ether were obtained.

### Preparation Example 2

Into a 2 liter glass flask, 317 g of 3,5,5-trimethylhexanol, 216 g of 1,4-dibromobutane, 14.3 g of tetrabutylammonium bromide and 442 g of a 52% by mass aqueous solution of sodium hydroxide were placed, and the reaction was allowed to proceed at 70° C. for 8 hours under stirring.

After the reaction was completed, the reaction mixture was transferred to a separatory funnel, and the aqueous phase was removed by filtration. The remaining organic phase was washed with 500 ml of water 5 times.

From the organic phase, 1,4-bis(3,5,5-trimethylhexoxy)butane was separated by distillation under a reduced pressure.

In accordance with similar reactions using 3,5,5-trimethylhexanol, 2-ethylhexanol, nonanol, decanol, 3,7-dimethyloctanol, octanol and a mixed alcohol of octanol and nonanol, 1,5-bis(3,5,5-trimethylhexoxy)pentane, 1,6-bis(3,5,5-trimethylhexoxy)hexane, 1,8-bis(3,5,5-trimethylhexoxy)octane, 1,8-bis(2-ethylhexoxy)octane, 1,5-bis(nonyloxy)-3-methylpentane, 1,5-bisdecyloxy)-3-methylpentane, 1,3-bis(decyloxy)-2,2-dimethylpropane, 1,6-bis(3,7-dimethyloctoxy)hexane, 1,4-bis(3,7-dimethyloctoxy)butane, 1,5-bis(octoxy)-3,3-diethylpentane and 1,5-bis(octoxy/nonyloxy)-3,3-diethylpentane were obtained.

### Preparation Example 3

Into a 2 liter glass flask, 96.2 g of tri(1,2-propylene glycol), 243.3 g of 1-bromononane, 14.3 g of tetrabutylammonium bromide and 442 g of a 52% by mass aqueous solution of sodium hydroxide were placed, and the reaction was allowed to proceed at 70° C. for 8 hours under stirring.

After the reaction was completed, the reaction mixture was transferred to a separatory funnel, and the aqueous phase was removed by filtration. The remaining organic phase was washed with 200 ml of water 5 times.

Water was removed from the organic phase using a vacuum evaporator until the liquid became transparent. The resultant liquid was filtered by pouring into a column packed with 40 g of silica gel, and an organic substance was obtained.

From the organic substance, 72 g of tri(2,3-propylene glycol) dinonyl ether was separated in accordance with the distillation under a reduced pressure.

In accordance with a similar reaction using 1-bromodecane, tri(2,3-propylene glycol) didecyl ether was obtained.

### Examples 1 to 15 and Comparative Examples 1 and 2

The properties which are the kinematic viscosity, the viscosity index the pour point and the evaporation loss of various types of base oils for lubricating oil compositions are shown in Table 1.

TABLE 1

| | | Number | Kinematic | | | | Evaporation |
|---|---|---|---|---|---|---|---|
| | | of carbon | viscosity | | | Pour | loss |
| | Base oil for | atoms in | (mm²/s) | | Viscosity | point | [120° C., 24 h] |
| | lubricating oil | entire oil | 100° C. | −20° C. | index | (° C.) | (% by mass) |
| Example | | | | | | | |
| 1 | C20-O-N4 | 24 | 2.253 | 93.25 | 120 | −47.5 | 1.45 |
| 2 | G16-O-N9 | 25 | 2.327 | 102.7 | 124 | −45 | 1.64 |
| 3 | G20-O-N5 | 25 | 2.360 | 102.9 | 127 | −47.5 | 1.57 |
| 4 | G16-O-N10 | 26 | 2.490 | 115.0 | 129 | −42.5 | 0.96 |
| 5 | G20-O-N6 | 26 | 2.496 | 114.9 | 128 | −45 | 0.62 |
| 6 | TMH-O-N8-O-TMH | 26 | 3.206 | 205.6 | 166 | −60> | 2.00 |
| 7 | G8-O-N8-O-G8 | 24 | 2.284 | 106.6 | 138 | −47.5 | 1.95 |
| 8 | N9-O-MC5-O-N9 | 24 | 2.361 | | 170 | −35.0 | |
| 9 | N10-O-NPG-O-N10 | 25 | 2.486 | 97.19 | 174 | −40 | 1.78 |
| 10 | DMC8-O-N6-O-DMC8 | 26 | 2.845 | 176.9 | 149 | −60> | 0.97 |
| 11 | DCM8-O-N4-O-DCM8 | 24 | 2.452 | 129.3 | 137 | −60> | 2.22 |
| 12 | N8-O-DEC5-O-N8 | 25 | 2.279 | 107.2 | 128 | −60> | 1.71 |
| 13 | N8/N9-O-DEC5-O-N8/N9 | 25˜27 | 2.457 | 126.7 | 128 | −60> | 1.08 |
| 14 | M92TPG | 27 | 2.529 | 95.4 | 178 | −60> | 1.31 |
| 15 | N102TPG | 29 | 2.913 | 126.1 | 185 | −50.0 | 0.85 |
| Comparative | | | | | | | |
| Example | | | | | | | |
| 1 | DOS | 26 | 3.247 | 232 | 150 | −50> | 0.99 |
| 2 | NPG-C8 | 13 | 2.336 | 113.1 | 126 | −50> | 2.45 |

### Examples 16 and 17 and Comparative Example 3

The results of the test of hydrolysis of lubricating oil compositions shown in Table 2 are shown also in Table 2.

TABLE 2

| | | | Comparative |
|---|---|---|---|
| | Example 16 | Example 17 | Example 3 |
| Lubricating oil composition | | | |
| (% by mass) | | | |
| G20-O-N8 | 99.0 | 0.0 | 0.0 |
| N8-O-DEC5-O-N8 | 0.0 | 99.0 | 0.0 |
| DOS | 0.0 | 0.0 | 99.0 |
| antioxidant *¹ | 1.0 | 1.0 | 1.0 |
| Decrease in weight of copper | 0.0 | 0.0 | 0.4 |
| catalyst (mg) | | | |
| Increase in total acid value of | smaller | smaller | 0.49 |
| oil layer (mg KOH/g) | than 0.01 | than 0.01 | |

*¹ A mixture of octadecyl 3-(3,5-di-t-butyl-4-hydroxyphenyl)propionate and 4,4-dioctyldiphenylamine each in an amount of 0.5% by mass

### Example 18

The result of the measurement of the volume resistivity of the lubricating oil composition shown in Table 3 is shown also in Table 3.

TABLE 3

| | | Volume |
|---|---|---|
| | | resistivity |
| | Composition | (Ω · cm) |
| Example 18 | N8-O-DEC5-N8 + 0.5% by mass of | 5 × 10⁸ |
| | electrically conductive additive *² | |

*² Sorbitan monooleate

As shown in Table 1 among the lubricating oil compositions of the present invention, the lubricating oil compositions of Examples 1, 4, 5 and 13 completely satisfied the required properties, i.e., a kinematic viscosity of not more than 140 mm²/s at −20° C., a viscosity index of at least 100, a pour point of not more than −40° C. and an evaporation loss of not more than 1.5% by mass, and were shown to be preferable base oils.

In Comparative Example 1, the kinematic viscosity at −20° C. was extremely great. In Comparative Example 2, the evaporation loss was great.

As shown in Table 2, the lubricating oil compositions of Comparative Examples showed the property of hydrolysis inferior to that of the lubrication oil compositions of Examples. As shown in Table 3, the volume resistivity was small in Example 16.

### INDUSTRIAL APPLICABILITY

The lubricating oil composition for fluid dynamic bearings of the present invention comprises a compound selected from specific monoether compounds, diether compounds, triether compounds and tetraether compounds, exhibits a small viscosity, a great viscosity index, excellent fluidity at low temperatures, lower evaporability and excellent energy saving property and can be advantageously used for compact fluid dynamic bearings used under rotation at a great speed.

1. A lubricating oil composition for fluid dynamic bearings which comprises 50 to 100% by mass of an ether compound comprising at least one ether bond and having 11 to 34 carbon atoms as a base oil and has a kinematic viscosity of at least 2.2 mm²/s at 100° C.

2. A lubricating oil composition for fluid dynamic bearings according to claim 1, wherein the ether compound comprising at least one ether bond and having 11 to 34 carbon atoms has 1 to 4 ether bonds.

3. A lubricating oil composition for fluid dynamic bearings according to claim 2, wherein the ether compound comprising at least one ether bond and having 11 to 34 carbon atoms is a monoether compound represented by a general formula (I):
R¹—O—R² (I)
wherein R¹ represents a monovalent hydrocarbon group comprising a side chain and having 8 to 24 carbon atoms, and R² represents a monovalent hydrocarbon group having 3 to 10 carbon atoms.

4. A lubricating oil composition for fluid dynamic bearings according to claim 2, wherein the ether compound comprising at least one ether bond and having 11 to 34 carbon atoms is a diether compound represented by a general formula (II):
R³—O—R⁴—O—R⁵ (II)
wherein R³ and R⁵ each represent a monovalent hydrocarbon group, R⁴ represents a divalent hydrocarbon group, at least one of groups represented by R³, R⁴ and R⁵ has a side chain, and a number of carbon atoms in entire groups represented by R³, R⁴ and R⁵ is 11 to 34.

5. A lubricating oil composition for fluid dynamic bearings according to claim 2, wherein the ether compound comprising at least one ether bond and having 11 to 34 carbon atoms is a tri- or tetraether compound represented by a general formula (III):
R⁶—O—(R⁷—O—)ₙ—R⁸ (III)
wherein R⁶ and R⁸ each represent a monovalent hydrocarbon group, R⁷ represents a divalent hydrocarbon group, n represents an integer of 2 or 3, at least one of groups represented by R⁶, R⁷ and R⁸ has a side chain and a number of carbon atoms in entire groups represented by R⁶, R⁷ and R⁸ is 11 to 34.

6. A lubricating oil composition for fluid dynamic bearings according to any one of claims 1 to 5, which comprises at least one of phenol-based antioxidants and/or amine-based antioxidants.

7. A lubricating oil composition for fluid dynamic bearings according to any one of claims 1 to 6, which comprises a lubricity improver additive.

8. A lubricating oil composition for fluid dynamic bearings according to any one of claims 1 to 7, which comprises an electrically conductive additive.

9. A lubricating oil composition for fluid dynamic bearings according to any one of claims 1 to 8, wherein an evaporation loss after a heat treatment at 120° C. for 24 hours is not more than 1.5% by mass.

10. A lubricating oil composition for fluid dynamic bearings according to any one of claims 1 to 9, wherein a kinematic viscosity at a temperature of −20° C. is not more than 140 mm²/s and a pour point is not more than −40° C.

11. A lubricating oil composition for fluid dynamic bearings according to ally one of claims 1 to 10, wherein a volume resistivity is not more than 1×10¹⁰ Ω·cm in substantial absence of particles of metals or particles of metal oxides.
